(19) United States
(12) Patent Application Publication
(10) Pub. No.: US 2001/0046701 A1
(43) Pub. Date: Nov. 29, 2001

(54) Nucleic acid amplification and detection using microfluidic diffusion based structures

(76) Inventors: Thomas H. Schulte, Redmond, WA (US); Bernhard H. Weigl, Seattle, WA (US)

Correspondence Address: JERROLD J. LITZINGER, SENTRON MEDICAL, INC., 4445 LAKE FOREST DR., SUITE 600, CINCINNATI, OH, 45242, US

(21) Appl. No.: 09/865,093

(22) Filed: May 24, 2001

Related U.S. Application Data
(60) Provisional application No. 60/206,878, filed on May 24, 2000.

(51) Int. Cl. C12M 1/34; F27D 11/00

(52) U.S. Cl. 435/287.2; 219/385

(57) ABSTRACT

A device for performing polymerase chain reaction (PCR) amplification and detection using microfluidic diffusion-based structures. Fluid containing DNA to be amplified is cycled repeatedly across hot and cold zones to enhance the multiplication process. The invention is used in conjunction with other devices to perform both single and multiple target detection.

### CROSS REFERENCE TO RELATED APPLICATIONS

This patent claims benefit from U.S. Provisional Patent Application Ser. No. 60/206,878, filed May 24, 2000, which application is incorporated herein by reference.

### BACKGROUND OF THE INVENTION

1. Field of the Invention

This invention relates generally to microfluidic devices for performing analytical testing, and, in particular, to a device and method for performing nucleic acid amplification using microfluidic diffusion-based separation processes.

2. Description of the Related Art

Microfluidic devices have recently become popular for performing analytical testing. Using tools developed by the semiconductor industry to miniaturize electronics, it has become possible to fabricate intricate fluid systems which can be inexpensively mass produced. Systems have been developed to perform a variety of analytical techniques for the acquisition of information for the medical field. Microfluidic channels are generally defined as a fluid passage which have at least one internal cross-sectional dimension that is less than 500 μm and typically between about 0.1 μm and about 500 μm.

In microfluidic channels, fluids usually exhibit laminar behavior; that is, they allow the movement of separate fluidic streams next to each other within the channel without mixing, other than diffusion. For example, a sample solution, such as whole blood, and an extraction solution, such as a buffer solution, are introduced into a common microfluidic channel, and flow next to each other until they exit the channel. Smaller particles, such as ions or small parts of DNA, diffuse rapidly across the fluid boundaries, whereas larger particles (e.g., large pieces of DNA or small pieces of DNA attached to a larger particle) diffuse more slowly. Large particles of a diameter of roughly more than 2 μm show no significant diffusion within the time the two flowing streams are in contact.

The principle of laminar flow has been addressed in a number of patents which have recently issued in the field of microfluidics. U.S. Pat. No. 5,716,852, which is incorporated herein in its entirety, is directed to a device, known as a T-Sensor, having a laminar flow channel and two inlet stream means in fluid communication with the laminar flow channel, which has a depth sufficiently small to allow particles from one stream to diffuse into the other stream. U.S. Pat. No, 5,932,100, which is also incorporated by reference herein in its entirety, is directed to a microfabricated extraction system for extracting desired particles from a sample stream. This device, known as an H-Filter, contains a laminar flow extraction channel and two inlet stream means connected to the extraction channel, with separate outlets at the exit of the extraction channel for a product stream containing the extracted particles and a by-product stream containing the remainder of the sample stream.

Recently, a number of protocols, test kits, and cartridges have been developed for conducting analyses on biological samples for various diagnostic and monitoring purposes. Immunoassays, agglutination assays, and analyses based on polymerase chain reaction (PCR), various legend-receptor interactions, and differential migration of species in a complex sample have all been used to determine the presence or concentration of various biological compounds or contaminants, or the presence of particular cell types.

PCR is a method which has been devised for amplifying one or more specific nucleic acid sequences or a mixture thereof using primers, nucleotide triphosphates, and an agent for polymerization, such as DNA polymerase. The extension produced of one primer, when hybridized to the other, becomes a template for the production of the desired specific nucleic acid sequence, and vice versa. The process is repeated as often as necessary to produce the desired amounts of the sequence.

The basic process for amplifying any desired specific nucleic acid sequence contained in a nucleic acid or mixture thereof is described in U.S. Pat. No. 4,683,202, in which a strand of DNA is copied using a polymerase. The process comprises treating complimentary strands of nucleic acid with two primers, for each specific sequence being amplified, under conditions such that for each different sequence being amplified an extension product of each primer is synthesized which is complimentary to each nucleic acid strand, wherein the primers are selected so as to be substantially complimentary to different strands of each specific sequence such that the extension product synthesized from one primer, when it is separated from its complement, can serve as a template for synthesis of the extension product of the other primer. The primer extension products are then separated from the templates on which they were synthesized to produce single-stranded molecules. Finally, the single-stranded molecules that are generated are treated with the primer generated under conditions such that a primer extension product is synthesized using each of the single strands as a template. This process is repeated until the desired level of sequence amplification is obtained.

U.S. Pat. No. 4,683,202, which issued Jul. 28, 1987, is directed to the PCR process for amplifying any desired specific nucleic acid sequence contained in a nucleic acid or mixture thereof. In an example disclosed therein, a solution was prepared which was heated to 100° C. for four minutes and allowed to cool to room temperature for two minutes, whereupon DNA polymerase was added and the cycle of heating, cooling, adding polymerase, and reacting was repeated many times. U.S. Pat. No. 5,939,291 is directed to an isothermal method of nucleic acid amplification which incorporates nonthermal means for denaturing the target nucleic acid or resultant amplification products, which enables the avoidance of the use of a thermal cycler component of any amplification equipment. The process can also be used in the context of a microfluidic device.

Other devices which are directed to microfluidic or microscale devices are: U.S. Pat. No. 5,916,776, which generates copies of a first strand of nucleic acid to generate copies of a second strand, and moves the copies of the second strand to a second location; U.S. Pat. No, 6,057,149, which employs silicon-based microscale microdroplet transport channels wherein the discrete droplets are differentially heated and propelled through stated channels; and U.S. Pat. No. 6,117,634, in which novel sequencing reactions using double-stranded templates are contemplated to take place in microfabricated reaction chambers. U.S. Pat. No. 5,333,675 teaches a device designed for performing automated amplification of nucleic acid sequences and assays using heating and cooling steps.

U.S. Pat. No. 5,955,029 is directed to devices for amplifying a preselected polynucleotide in a sample by conducting a polynucleotide polymerization reaction. The device may be used to implement a PCR in the reaction chamber, which is provided with the sample polynucleotide, polymerase, nucleotide triphosphates, primers and other reagents required for the PCR, and contains means to thermally control the temperature of the contents of the reaction chamber to dehydridize double stranded polynucleotide, to anneal the primers, and to polymerize and amplify the polynucleotide. U.S. Pat. No. 5,965,410 discloses a device for controlling process parameters, including fluid temperature, of a system by the application of electric current to the material such that the material can be successively heated and cooled for biological applications such as PCR.

U.S. Pat. No. 6,210,882 is directed to a method for performing rapid and accurate thermocoupling on a sample for performing PCR within microchannels on a microchip using a non-contact heat source. Positive cooling is accomplished by use of a non-contact cooling source directed at the vessel containing the sample. Cooling, like heating, can be accomplished through any member of steps, with a different temperature of steps, with a different temperature being maintained at each step.

Methodologies using PCR for diagnostic purposes are well established. PCR amplification has been used for the diagnosis of genetic disorders, and generation of specific sequences of closed double standard DNA for use as probes and to create larger amounts of DNA for sequencing.

Thus, a need has been created for convenient economical systems for PCR analyses, which could be used in a wide range of potential applications in clinical tests, such as test for paternity, genetic and infectious diseases.

### SUMMARY OF THE INVENTION

It is therefore an object of the present invention to provide a device for amplifying a preselected polynucleotide in a sample by conducting a polynucleotide polymerization reaction.

It is a further object of the present invention to provide a compact, single use module capable of analyses involving polymerase chain reaction (PCR) that is economical to manufacture and use.

These and other objects are accomplished with a device which comprises a substrate microfabricated to define a sample inlet port and a mesoscale flow system extending from the inlet port. The mesoscale flow system includes a polynucleotide polymerization reaction chamber in fluid communication with the inlet port which is provided with reagents required for polymerization and amplification of a preselected polynucleotide.

### BRIEF DESCRIPTION OF THE DRAWINGS

FIG. 1 is a diagram showing non-contact thermal heating of a fluid in a microscale channel;

FIG. 2 is a diagram showing the thermocycling in PCR according to the present invention;

FIG. 3 is a diagram showing PCR using single target amplification and detection according to the present invention; and

FIG. 4 is a diagram showing PCR using multiple target detection according to the present invention.

### DETAILED DESCRIPTION OF THE PREFERRED EMBODIMENTS

FIG. 1 is a diagram showing a method of heating a fluid plug within a microfluidic channel. A fluid plug 2 is contained within a microfluidic channel which traverses a pair of heat pads 6, 8. Fluid plug 2 can be cycled back and forth within channel 4 until it reaches a desired temperature over heat pads 6, 8.

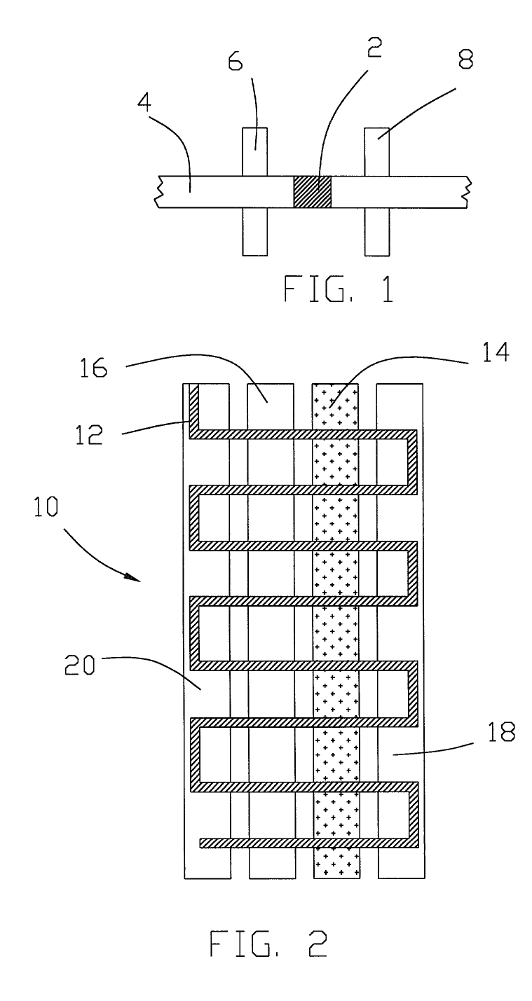

Referring now to FIG. 2, there is shown a microfluidic device for performing PCR, generally indicated at 10. Device 10 includes a microfluidic flow channel 12, a pair of heat pads 14, 16, and a pair of cooling regions 18, 20. Channel 12 consists of a sinuous S-shaped pathway which traverses across heat pads 14, 16 and cooling sections 18, 20. In this arrangement, the contents of channel 10, which consists of Taq-polymerase, dNTP and two DNA primer sequences which are flowing laminarly within channel 12 alongside the sample containing the DNA to be amplified, can be cycled repeatedly across hot and cold zones which is necessary for the amplification of the described DNA region of interest. Heat pads 14, 16 can be manufactured from anything that conducts and/or stores heat, such as metal plates, vices, or hot water. Joule heating or radiation heating may also be used. Typical temperature for pads 14,16 generally can be around 95° C., and around 45 to 50° C. for cooling regions 18, 20.

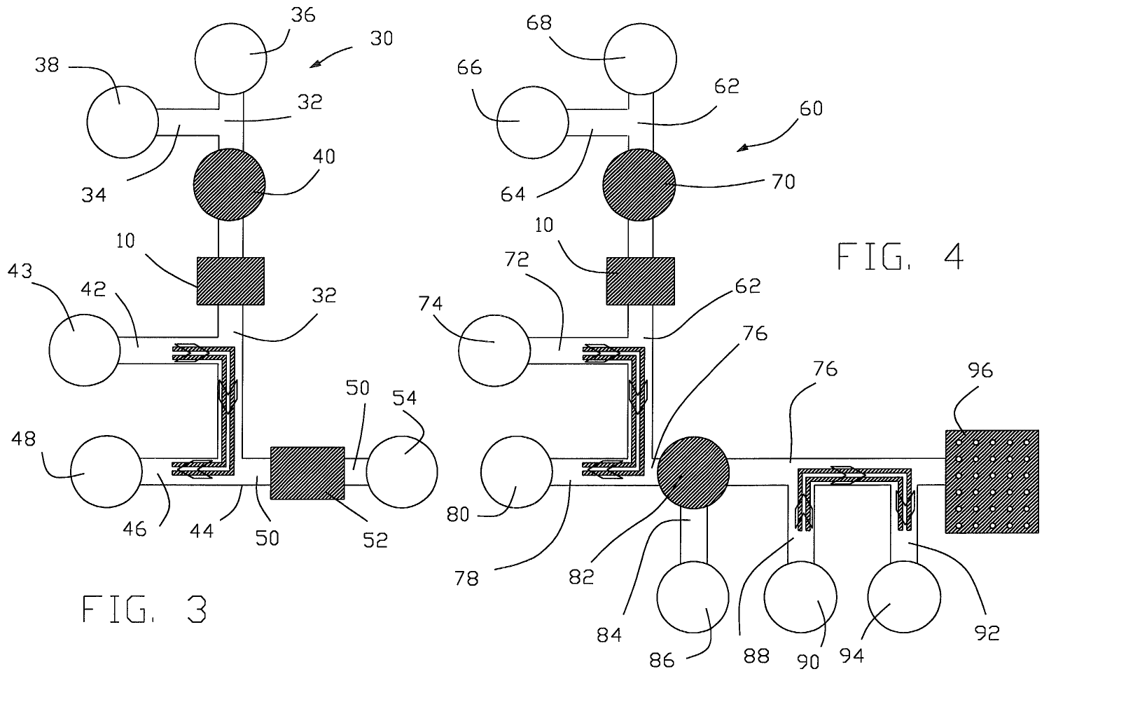

One embodiment of PCR involving single-target amplification and detection is shown in FIG. 3. Referring now to FIG. 3, a PCR amplification system, generally designated at 30, contains a main channel 32 and an intersecting channel 34. A first port 36 is coupled to the inlet of channel 32, while a second port 38 is coupled to the inlet of channel 34. Main channel 32 is connected to a mixing structure 40, which mixer is preferably of the type described in U.S. patent application Ser. No. ______, which application is hereby incorporated by reference in its entirety. However, any mixing structure which provides sufficient mixing may be used. The output of mixer 40 is coupled to PCR thermocycler 10, which is shown and discussed in detail with respect to FIG. 2.

Main channel 32 exits thermocycler 10 and is intersected by a second intersecting channel 42 having an input port 43. Downstream from channel 42, channel 32 terminates in an exit channel 44. Exit channel 44 contains a waste section 46 having a port 48, and a sample section 50. Section 50 is coupled to a detection means 52. The output of detection means 52 is coupled to an output port 54 via section 50.

The structure formed by channel 42, main channel 32, channel 46 and channel 50 operates in the same manner as the absorption enhanced differential extractor device which is described in detail in U.S. Pat. No. 5,971,158, which patent is hereby incorporated by reference in its entirety. This device, which is commonly referred to as an “absorption-enhanced Hfilter”, is useful for extracting desired particles from a sample stream containing the desired particles. A sequestering material within the extraction channel captures the desired particles in the extraction stream.

In operation, a sample containing DNA is loaded into port 36, while a sample containing Taq polymerase, a primer 1, and a primer 2 is loaded into port 38. Primer 1 preferably consists of large particles or may be attached to larger molecules or particles, while Primer 2 preferably consists of labeled particles. These two substances travel through channel 32 in a laminar fashion where diffusion takes place, as previously discussed, until the streams reach mixer 40, where the substances are combined to form an essentially homogeneous mixture. This mixture flows from mixer 40 to thermocycler 10, where conventional PCR amplification is performed in the mixture using the structure shown in FIG. 2. In the present embodiment, the last PCR cycle is ended at the low temperature as DNA is attached to the primers. The output stream of thermocycler 10 flows in main channel 32 and contains multiple copies of DNA attached to labeled primer molecules, as well as excess primer 1 and primer 2.

An extraction solution containing primer absorbing particles is loaded into port 43 and flows through channel 42 to main channel 32 where it contacts and flows next to the output stream from thermocycler 10, without mixing other than diffusion. In this embodiment, the absorbing particles in the solution from channel 42 remove fast-diffusing labeled primer molecules from equilibrium. The length of channel 32 between thermocycler 10 and channel 44 is chosen such that essentially all labeled primer molecules have diffused across the laminar flow boundary between the fluids.

As the contents of channel 32 reach channel 44, the extraction solution from channel 42 now contains a waste product containing primer absorbing particles, primer 1 molecules, and other small molecules as a result of diffusion. This stream exits channel 32 by way of section 46 of channel 44, and flows into exit port 48, while the stream which contains particles of interest exits channel 32 by way of section 50 of channel 44, and flows to detection means 52. In the present embodiment, detection means 52 is preferably a fluorescent detector. The stream now contains multiple copies of the desired DNA, and exits device 30 via port 54.

An embodiment showing multiple target detection is shown in FIG. 4. Referring now to FIG. 4, a PCR amplification system, generally designated at 60, contains a main channel 62 and an intersecting channel 64. A first port 66 is located at the end of channel 64 opposite to its intersection with channel 62, while a port 68 is located at the end of channel 62 opposite its intersection with channel 64. Main channel 62 is connected to a mixing structure 70, which mixer is preferably of the type shown in FIG. 2 and also described in U.S. patent application Ser. No. ______, but may consist of any suitable mixing device. Mixer 70 receives the contents of channels 62 and 64 which flow in a laminar fashion, and provides an essentially homogeneous mixture to PCR thermocycler 10, which has previously been described with respect to FIGS. 2 and 3.

Channel 62 exits thermocycler 10 and is intersected by a channel 72 which extends from an input port 74. Channel 62 continues downstream where it terminates at a crossing channel 76. Channel 76 is comprised of a waste section 78 which terminates in an exit port 80. Channel 76 is connected at its other end to a mixing/heating structure 82, while a channel 84 which terminates at a port 86 is also coupled to mixer 82. Channel 76 exits mixer 82 where it is coupled to an intersecting channel 88 coupled to a port 90. Channel 76 continues along past channel 88 where it intersects a waste channel 92 coupled to a waste port 94. Channel 76 finally terminates at a detecting device 96.

In operation, multiple target amplification and detection is performed by loading a sample containing DNA into port 68. A mixture of Taq polymerase, primer 1 and primer 2 is loaded into port 66. These primers in this mixture are intended for multiple targets, and are roughly the same size, with none of the particles very large. The mixture loaded into port 66 flows within channel 62 where it flows laminarly with the sample containing DNA which was loaded into port 68. The contents of channel 62 enter mixing structure 70, and exit mixture 70 as an essentially homogeneous fluid.

The mixed fluid enters PCR thermocycler 10 where DNA amplification occurs using the PCR method. The last PCR cycle performed by thermocycler 10 is ended at high temperature as the DNA is detached from the primers within the fluid mixture. The flow stream exiting thermocycler 10 now contains multiple copies of DNA detached from primer molecules, as well as excess primer 1 and primer 2 for multiple targets.

An extraction solution containing primer absorbing particles for primers 1 and 2 for each targeted DNA piece is loaded into port 74, where it flows through channel 72 into main channel 62, where it contacts with the flow stream exiting thermocycler 10 in a laminar fashion. The combined fluid stream flows through channel 62, where the primer absorbing particles remove fast-diffusing primer molecules from equilibrium. After sufficient time and travel within channel 62, almost all primer molecules are removed from system 60 by passing through waste channel 78 into waste port 80. Waste port 80 contains primer absorbing molecules, primers 1 and 2 for multiple targets and other small molecules, all of which have diffused across channel 62. The remaining fluid from channel 62 passes into crossing channel 76, where it enters mixing/heating structure 82. Also flowing into structure 82 is a fluorescent labeled primer 1 for each of the targeted DNA sequences, which are loaded into port 86. Structure 82 both mixes the two fluids and heats the solution to annealing temperature, which is approximately 96° C. This process opens up the strands of DNA within structure 82 and are passed along within channel 76.

An extraction solution containing primer-absorbing particles is loaded into port 90, and flows within channel 88 to channel 76, where it flows laminarly adjacent to fluid exiting structure 82. As the flow reaches waste channel 92, waste containing primer absorbing particles, primers 1 and other small molecules which have diffused across the laminar boundary exits channel 92 and flows into port 94, while the remaining flow within channel 76 which now contains multiple copies of DNA of multiple targets attached to labeled primers 1.

The flow from channel 76 enters fluorescent detector structure 96, where primers 2 for multiple targets are immobilized on the bottom of structure 96, while the various DNA targets, each labeled with a fluorescent primer 1, attach to a specific site on structure 96 and can therefore be identified and quantified.

The structure of device 60 after thermocycler 10 operates in the same manner as two absorption enhanced differential extractor devices, which were discussed previously, which are operating in series.

The PCR assays performed using the present invention can be used in a wide range of applications such as the generation of specific sequences of cloned double-stranded DNA for use as probes, the generation of probes is specific for uncloned genes by selective amplification of particular segments of cDNA, the generation of libraries of cDNA for sequencing, and the analysis of mutations.

While the present invention has been shown and described in terms of several embodiments thereof, it will be understood that this invention is not limited to these particular embodiments and that many changes and modifications may be made without departing from the true spirit and scope of the invention as defined in the appended claims.

What is claimed is:

1. A device for sequentially heating and cooling a fluid, comprising:
a microfluidic channel having a first and a second end;
a fluid specimen located within said channel having a first temperature;
a first region of higher temperature than said first temperature located between said first and second ends of said channel;
a second region of lower temperature than said first temperature located between said first and second ends of said channel
whereby said fluid specimen flows through said channel the temperature of at least a portion of said fluid is sequentially increased and lowered.

2. The device of claim 1, wherein said first region comprises a heat strip.

3. The device of claim 1, comprising a plurality of first and second regions located between said first and second ends.

4. The device of claim 1, wherein said first region is spaced apart from said channel.

5. A device for sequentially heating and cooling a fluid, comprising:
a microfluidic channel having a first and second end;
and a heating element placed in proximity to said channel,
such that said heating element increases the temperature of portions of said channel at multiple discrete locations.

6. The device of claim 5, further comprising a cooling element placed in proximity of said channel such that said cooling element decreases the temperature of portions of said channel at multiple discrete locations.

7. The device of claim 1, wherein said first region of higher temperature comprises approximately 95° C.

8. The device of claim 1, wherein said second region of lower temperature comprises between 45 and 50° C.

9. The device of claim 1, wherein said first region of higher temperature comprises a metal plate.

10. The device of claim 1, wherein said first region of higher temperature comprises a radiation heater.

11. The device of claim 1, wherein said first region of higher temperature comprises joule heating.

12. A microfluidic device for performing a polymerase chain reaction to amplify selected nucleic acid sequences, comprising:
a first microfluidic channel containing a selected nucleic acid sequence;
a second microfluidic channel containing substances necessary to perform nucleic acid amplification;
a main microfluidic channel having a sinuous pathway, coupled to said first and second channels such that the contents of said first and second channels establish a laminar flow within said main channel of an initial temperature such that particles may diffuse across the laminar boundary;
a first region of higher temperature than said initial temperature located along said sinuous pathway of said main channel;
a second region of lower temperature than said initial temperature located along said sinuous pathway of said main channel spaced apart from said first region;
whereby the laminar flow within said main channel sequentially passes said first region and said second region such that the temperature of the laminar flow within said main channel is sequentially increased and lowered to amplify the selected nucleic acid sequences.

13. The device of claim 12, wherein said substances necessary to perform nucleic amplification include Taq-polymerase.

14. The device of claim 13, wherein said substances further include dNTP and two DNA primer sequences.

15. The device of claim 12, wherein said main channel is S-shaped.

16. The device of claim 12, comprising a plurality of first regions and a plurality of second regions, wherein the locations of said first and second regions are alternated.
